(19) United States
(12) Patent Application Publication
(10) Pub. No.: US 2026/0232498 A1
(43) Pub. Date: Aug. 13, 2026

(54) Tag Attachment

(71) Applicant: Melissa Harris, Ashburn, VA (US)

(72) Inventor: Melissa Harris, Ashburn, VA (US)

(21) Appl. No.: 19/048,074

(22) Filed: Feb. 7, 2025

(51) Int. Cl. A61F 13/34

(52) U.S. Cl. CPC A61F 13/34

(57) ABSTRACT

A tag attachment having a lemniscate shape which can be securely attached to the tampon string of a tampon by encasing the tampon string with the tag attachment. The tag attachment has a top side provided with an adhesive layer and a peelable and removable protective layer applied over the layer of adhesive material. The tag attachment is manually secured to the tampon string by means of the layer of adhesive material and encased therein by folding the lemniscate shaped attachment onto itself to adhesively secure and firmly encase the tampon string within the tag attachment. The tag attachment extends and freely hangs outside the vaginal canal, thereby enabling the user to efficiently and effectively control and interact with the tampon string. In one embodiment, the length of the tampon string may be shortened.

### BACKGROUND OF THE INVENTION

In the United States, approximately forty two percent of females use tampons to manage their feminine hygiene during their menstrual cycle, using an average of 11,000 to 16,000 tampons in their lifetime. A tampon is a menstrual product designed to be inserted through the vagina opening to be placed temporarily in the vaginal canal to absorb blood and vaginal secretion of the female body during a menstrual cycle. Attached to the tampon's base is a soft, flexible, and long string (henceforth referred to as “string”) comprised of interwoven threads. It typically measures an average between 3 to 5 inches in length and a few millimeters in diameter. The string extends and freely hangs outside the vaginal canal after the tampon has been inserted, allowing users to locate and grasp it by using their thumb and finger(s) to safely and efficiently remove the tampon from the vaginal canal.

Following the insertion of the tampon into the vaginal canal, the string's limited length and thin diameter (henceforth referred to as “limited size”) present several disadvantages for users as it extends and freely hangs outside the canal. It complicates users'action of shortening the string for concealment and locating and grasping the string to maneuver it efficiently during bathroom use and tampon removal while they sit or squat over a toilet with their knees apart, stand with one leg raised, or lie down, typically in a public restroom, private bathroom, or shared or private space. These activities can be challenging to accomplish as users may have physical limitations or experience potential issues with balance and stability, particularly when there are limited sources of support available. Consequently, due to its disadvantages, users encounter difficulties and challenges when locating, handling, and shortening the string as further explained below:

A significant disadvantage of the string is its limited size which complicates users'action of promptly and effectively locating it by visual or tactile identification. This challenge arises from the string's small dimensions as it hinders users'ability to detect it quickly and efficiently. Its limited size also makes it more prone to movement as friction occurs between the string and the surrounding skin from physical movements, such as walking, running, and sitting, causing it to shift or become obscured within the genitalia and anal areas making it difficult to locate.

Due to these challenges, users perform various actions to locate the string by re-positioning their bodies, using a mirror, or blindly searching their genitalia and anal areas by hand. To illustrate, it can become nestled between the inner and outer folds of the labia, which obscures the view of the string. This requires users to reposition their bodies and use their hands to gently spread apart the folds to examine each fold to locate the string. Similarly, it can become twisted or entangled in pubic hair, where it becomes hidden from view. This requires users to use their hands to delicately move or divide pubic hair or use a mirror to locate the string. Also, it can be positioned near the anus, which obstructs the view of the string. This requires users to reposition their bodies and use their hands to gently spread apart the skin around the anus to locate the string. Likewise, the string can be drawn into the vaginal canal, where it cannot be immediately detected. This requires users to insert their fingers into the canal to feel along the vaginal walls to locate the string, which could increase the risk of introducing bacteria or other contaminants into the canal, potentially leading to an infection. Moreover, the angle at which the string is positioned inside the vaginal canal may not allow for easy detection. This raises the concern that it may be lost within the vaginal canal, leading to users requiring immediate medical assistance or the assistance of another individual to locate the string.

Additionally, users searching for the string may have unintended contact with bodily fluids, such as blood, urine, feces, and secretions. This results in users hesitating to use their hands to locate the string within these areas. Moreover, users may be reluctant to feel around these areas when skin conditions, such as skin abrasions or inflammation exist, as it can aggravate or worsen the sensitivities. Consequently, these actions prolong the time it takes to locate the string, causing users delays, discomfort, anxiety, and frustration.

The string's limited size also prevents users from grasping it effectively, as it provides less surface area for users to achieve and maintain a firm grip using only their thumb and finger(s). This hinders users'ability to apply sufficient pressure to grasp the string, increasing the likelihood of it slipping or being mishandled during use. Additionally, it is particularly difficult for users with physical limitations as they may experience difficulty exerting enough pressure to achieve a firm grip. The challenges of grasping the string are further compounded when it absorbs bodily fluids when displaced within the genitalia areas as the texture becomes slippery and wet. This reduces friction between the fingers and the string, complicating the ability to maintain a secure grip. Furthermore, due to its limited size, users encounter difficulty in confirming whether they have securely grasped the string. This arises because it is challenging for users to properly feel the string between their fingers, leading to uncertainty about their grip.

Due to these challenges, users perform various actions to grasp the string. One action is to wind the string around their thumb and finger(s) or hook their finger(s) under it to achieve a secure grip; however, it creates excessive pressure on the thumb and finger(s), causing discomfort, pain, and skin irritation. This action can also inadvertently displace the tampon when force is exerted on the string, which leads to unintended pulling and tugging on the tampon, causing it to shift downward in the vaginal canal. Moreover, an alternative action involves users wrapping or folding tissue products such as toilet paper, paper towels, and tissue around the string to achieve a secure grip and reduce direct contact with the genitalia and anal areas. These precautions are often taken due to the potential discomfort and unease associated with touching the genitalia and anal areas where bodily fluids may be present. Consequently, these actions contribute to prolonged delays, heightened uncertainty, and increased frustration for users when grasping the string.

The challenges in grasping the string, exacerbated by its limited size, hinder users'ability to handle and control its movements. This hinders users'ability to maneuver the string efficiently to remove the tampon from the vaginal canal and to position it away from streaming bodily fluids during bathroom use. This is further complicated when users use various actions to grasp the string as it shortens the usable length, limiting their ability to handle it. Consequently, these challenges impede users'ability to efficiently maneuver the string to adjust its position to perform these actions without experiencing complications.

Additionally, the restriction of handling the string results in unintended force exerted on the tampon as users attempt various actions to handle and control its movements without complication. This can lead to pulling or tugging on the tampon, potentially causing it to become displaced within or to fall out of the vaginal canal. The displacement of the tampon often necessitates immediate removal due to discomfort and potential irritation caused by the tampon shifting downward within the canal. As a result, users who did not intend to remove their tampons may need to do so abruptly. In both these instances, it can cause issues as users may not be adequately prepared and lack the necessary items, such as toilet paper or sanitary disposal bags, to dispose of their tampons immediately and cleanly. Consequently, the lack of preparation and necessary items for disposal can lead to bodily fluids from the tampon contacting users'hands or clothing, causing discomfort and increased cleanup.

Moreover, the limitation of handling the string impedes users who keep their tampons inserted during bathroom use from effectively maneuvering it away from the urethra and anus openings while urinating or defecating. Its limited length and maneuverability make it difficult for users to handle the string while maintaining a sufficient distance from these openings, resulting in the string becoming wet and unintended contact with bodily fluids. This poses difficulties for users who choose not to change their tampons after bathroom use and continue to wear them, as a wet string can lead to hygiene concerns. The moisture on the string can harbor bacteria that may be present in the fluids, such as those that cause urinary tract infections. During continued wear, physical movements may cause the string to shift and rest on other parts of the genitalia and anal areas, increasing the risk of bacteria spreading and potentially causing cross-infection. Additionally, a wet string can cause dampness or staining of undergarments, leading to unpleasant odors and discomfort.

Additionally, its limited length and maneuverability make it challenging for users who prefer minimal contact with touching their genitalia area. This limitation makes it difficult to employ various actions to control the string's movements to avoid contact with this area. This is particularly challenging for users concerned with hygiene, discomfort, and contact with fluids or existing skin conditions. Prolonged contact with skin conditions can worsen sensitivities, resulting in irritation and discomfort during handling.

Due to these challenges, users perform various actions to handle the string as effectively as possible. One action is for users to adjust their body positions or assume awkward postures to maneuver the string with minimal difficulty. This involves users using one hand to grip and maneuver the string at a comfortable angle and position while the other is used for balance, such as leaning on a nearby surface. This action requires users to concentrate heavily on stabilizing and controlling the string, maintaining balance, avoiding direct contact with the genitalia area, and preventing unintended force on the string to achieve optimal maneuverability with the limited usable length. This poses challenges for users as they may experience difficulties in maintaining steadiness which increases the likelihood of the string, hands, and even legs coming into contact with bodily fluids, especially when the tampon falls out of the vaginal canal.

An alternative action used to handle the string to maneuver it away from the urethra and anus openings while urinating or defecating to prevent it from becoming wet is users may choose to tuck the string inside the vaginal canal or in between the skin folds of the labia. This poses challenges and can result in complications, as tucking the string within these areas makes it difficult to locate and access. Also, when it is tucked between the skin folds of the labia, it can cause irritation and discomfort. Likewise, when the string is tucked inside the vaginal canal, users may forget about its presence, potentially leading to extended tampon wear. This increases the risk of a severe but rare condition called toxic shock syndrome. Additionally, tucking the string inside the vagina may lead to difficulty retrieving the tampon, especially if it becomes deeply embedded or entangled. Consequently, these actions affect users' ability to handle the string effectively, resulting in delays, discomfort, and complications.

Another disadvantage of the string is that it may protrude beyond users'clothing and undergarments, such as swimwear, bodysuits, lingerie, and similar clothing. This poses a significant likelihood of unintentional exposure, particularly in public settings, where exposure can lead to discomfort and self-consciousness. The discomfort of dealing with a protruding string can limit users' choices in clothing, as they may avoid wearing certain styles that could reveal the string.

Due to this challenge, users need to trim or knot the string to shorten it so it can be concealed. This poses challenges as it requires precise measurement to ensure the string is not cut too short while leaving enough length to be located, handled, and shortened. Users may encounter additional difficulties as they may not have access to the tools needed, such as scissors to shorten the string. Alternatively, users may knot the string; however, this also presents challenges, as the limited length of the string makes it difficult to tie it into a tight and secure knot. This often leads the string to unravel during wear and become exposed. As a result, this action can significantly impact users' comfort and confidence in managing their menstrual hygiene discreetly.

### SUMMARY OF THE INVENTION

The tag attachment of the present subject matter has a lemniscate shape which is designed to be manually attached to the end of an existing tampon string (henceforth referred to as “string” in this section) to enable a user to efficiently and effectively control and interact with the string. The tag attachment streamlines user interaction by making it easier to locate and handle the string during bathroom use to prevent it from becoming soiled with bodily fluids or to facilitate tampon removal. Additionally, the design includes a feature that allows users to shorten the string for discreet concealment using the tag attachment. Furthermore, the tag attachment is designed to be superior to the previous tampon string accessory apparatuses by focusing on enhanced usability, accessibility, functionality, user experience, and a more intuitive design. The tag attachment incorporates ergonomic attributes to optimize ease of use, efficiency, effectiveness, inclusiveness, and comfortability, enhancing users'ability to locate, handle, and shorten the string.

The tag attachment of the present subject matter significantly improves prior solutions by streamlining the process of affixing an accessory to a string, simplifying the task for users and resulting in an enhanced user experience. The tag attachment is designed to allow the user to effortlessly affix it directly to the string. The tag attachment addresses shortcomings of the previous solutions with a design that affixes the string at the center of its circular parts to ensure a comprehensive and strong bond. By eliminating the previous processes, the tag attachment's streamlined process enhances usability to ensure the attachment process is simpler, more efficient, and more effective to offer a superior user experience compared to those previous solutions.

Moreover, the tag attachment is designed to incorporate ergonomic attributes to optimize usability and accessibility by vastly improving the material, size, and shape to be superior to those previous solutions. Its material composition is a fibrous material that is soft, lightweight, and durable, which is a significant improvement over the previous solutions. The improved material composition of the tag attachment provides a comfortable tactile experience and ease of handling to enhance interaction and wear. Additionally, the tag attachment's ample-sized dimensions make it easily noticeable and identifiable as it freely hangs from the string, simplifying the process of locating it. The tag attachment is designed with a lemniscate-shaped, two-sided, and three-dimensional design that maximizes usability, accessibility, and functionality. The lemniscate shape is sleek, uniform, and compact optimizes its interaction with the body's curves to enhance comfort and efficiency when worn and handled while remaining unnoticeable.

Ultimately, the tag attachment is superior to prior efforts by effectively addressing their shortcomings. This is achieved by deliberately applying usability, accessibility, functionality, and user experience principles enhanced by ergonomic considerations. These improvements significantly streamline the process of affixing and interacting with the tag attachment to facilitate control and interaction with the string. This ensures a seamless user experience from start to finish. By setting a new innovative design, the tag attachment empowers users to locate and handle the string efficiently and effectively.

### BRIEF DESCRIPTION OF DRAWINGS

FIG. 1 is a perspective view of the tag attachment of the present subject matter, illustrating the tag attachment's lemniscate-shaped design.

FIG. 2 is a top view of the tag attachment of the present subject matter depicting a layer of adhesive material across the top side.

FIG. 3 is a top perspective view of the tag attachment of the present subject matter illustrating a peelable protective layer which is applied over the adhesive material.

FIG. 4 is a side perspective view of the tag attachment of the present subject matter, illustrating how the user holds the tag attachment to affix it to the tampon string.

FIG. 5 is a perspective view of the tag attachment of the present subject matter, illustrating how the protective layer covering the adhesive material is removed.

FIG. 6 is a top view of the tag attachment of the present subject matter, illustrating the user locating the specific point on the tampon string to affix the tag attachment.

FIG. 7 is a top perspective view of the tag attachment of the present subject matter, illustrating how the tag attachment folds in half onto itself, encasing the tampon string inside.

FIG. 8 is a perspective view of the tag attachment of the present subject matter, illustrating how once the tag attachment is folded in half, encasing the tampon string inside, the user applies pressure to secure a connection between the tag attachment and the tampon string.

FIG. 9 is a front perspective view of the tag attachment of the present subject matter, illustrating how once the tag attachment is affixed to the tampon string, the user can release the tag attachment and allow it to hang feely.

FIG. 10 is a front perspective view of the tag attachment of the present subject matter, illustrating how the user may position the tag attachment against their pelvic area, covering the attachment with their undergarments, to secure it from moving.

FIG. 11 is a front perspective view of the tag attachment of the present subject matter, illustrating how the user locates the tag attachment to remove the tampon.

FIG. 12 is a front perspective view of the tag attachment of the present subject matter, illustrating how the user applies pressure to the tag attachment to remove the tampon.

FIG. 13 is a top perspective view of the tag attachment of the present subject matter, illustrating how the user holds the tag attachment to affix it to the tampon string to shorten its length for concealment.

FIG. 14 is a top perspective view of the tag attachment of the present subject matter, illustrating how the end of the tampon string is folded over itself to form a single loop, and the point where it overlaps is affixed to the top side of the tag attachment.

FIG. 15 is a top perspective view of the tag attachment of the present subject matter, illustrating how the tampon string is secured to the adhesive side.

FIG. 16 is a top perspective view of the tag attachment of the present subject matter, illustrating how the tag attachment is affixed at the point where the tampon string overlaps, encasing the tampon string inside.

FIG. 17 is a top perspective view of the tag attachment of the present subject matter, illustrating how once the tag attachment is folded in half, encasing the tampon string inside, the user applies pressure to secure a connection between it and the tampon string.

FIG. 18 is a top perspective view of the tag attachment of the present subject matter, illustrating how once the tag attachment is affixed to the tampon string, the user can release the tag attachment to allow it to hang freely from the vaginal canal for discreet concealment.

### DETAILED DESCRIPTION OF THE INVENTION

In the following description, numerous specific details are set forth to clearly describe various specific embodiments disclosed herein. One skilled in the art, however, will understand that the presently claimed invention may be practiced without all of the specific details discussed below. In other instances, well known features have not been described so as not to obscure the invention. Additionally, like reference numbers are used to identify like elements. Furthermore, the drawings are intended to illustrate major features of exemplary embodiments in a diagrammatic manner. The drawings are not intended to depict every feature of every implementation nor relative dimensions of the depicted elements, and are not drawn to scale.

Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless limited otherwise, the terms “connected,” “coupled,” and “mounted,” and variations thereof herein are used broadly and encompass direct and indirect connections, couplings, and mountings. In addition, the terms “connected” and “coupled” and variations thereof are not restricted to physical or mechanical connections or couplings.

It is to be understood that the phraseology and terminology used herein is for the purpose of description and should not be regarded as limiting. The use of the terms “including”, “comprising”, “having”, and variations thereof herein is meant to encompass the items listed thereafter and equivalents thereof as well as additional items.

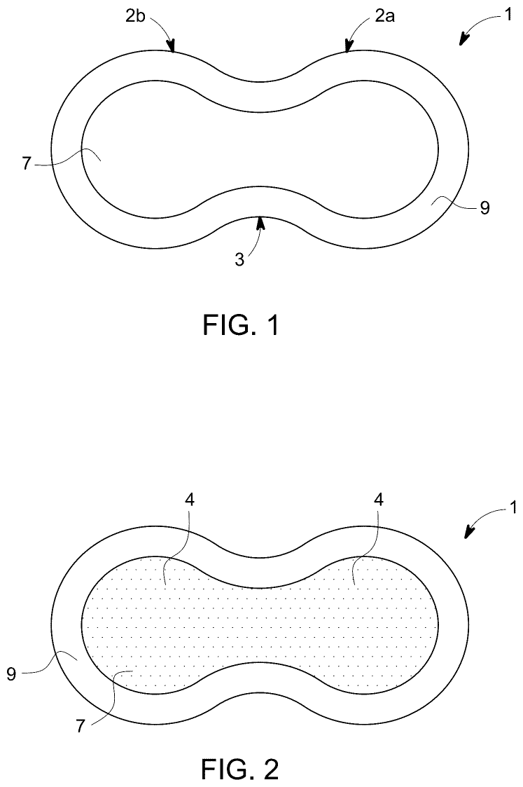

The following description and the drawings are illustrative of the invention but are not limiting. FIGS. 1 through 18 generally show various embodiments and components of the tag attachment of the present subject matter for attachment to a tampon string. The tag attachment 1 is preferably about one and a half inches in length and about half an inch in height but as will be obvious to those skilled in the art, it is not limited to this dimension. The tag attachment may be composed of any natural or man-made fibrous material that is soft, lightweight, and durable for comfort and functionality. Nonlimiting examples of such fibrous material include cotton, bamboo, cellulose made from wood pulp, lyocell, or other suitable materials. These materials may be waterproof, water-repellent, water-resistant, or coated to be waterproof, water resistant, or water resistant. As shown in FIG. 1-3, the tag attachment 1 is two-sided, three-dimensional, and preferably has a lemniscate-shape (also known as “figure eight-shape” or infinity). The lemniscate shape design of the tag attachment of the present subject matter provides the optimal configuration to handle the tag attachment 1 by hand.

Referring now to FIG. 1, the tag attachment 1 consists of two symmetrical circular parts 2a and 2b connected by a flexible midsection 3 in the flat position. In this way, the midsection 3 provides a movable connection between the first circular part 2a and the second circular part 2b, allowing them to evenly fold from an open-to-close position (transitioning from 1 to 180 degrees). As shown in FIG. 2, the tag attachment 1 has a top side 7, a bordered edge 9, and a layer of adhesive material 4, as shown as the dotted area 4. Preferably, the adhesive material spans across the top side 7 and does not extend beyond the bordered edge 9 or the entire surface area of the tag attachment 1. The adhesive material 4 used on the tag attachment 1 preferably is formulated to offer sufficient bonding to keep the tag attachment 1 secured to a tampon string 8 while allowing for repositioning on the tampon string as necessary. Nonlimiting examples of adhesive materials suitable for use in the tag attachment of the present subject matter include pressure-sensitive adhesive, polyolefin-based hot melt adhesive, and other similar adhesives as will be obvious to those skilled in the art.

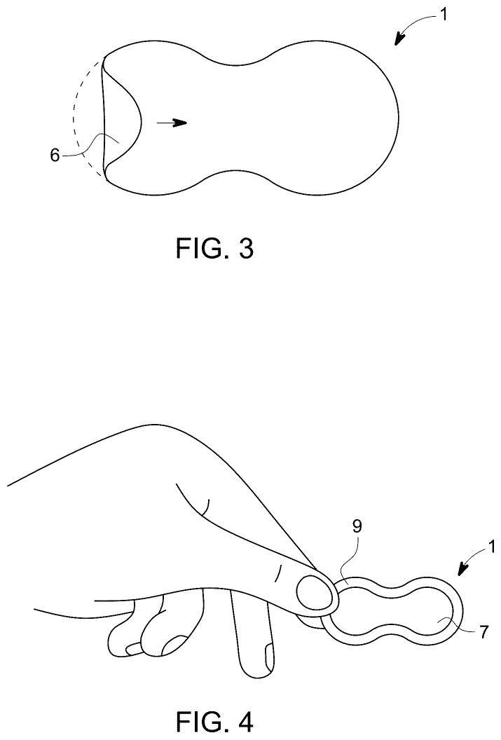

As shown in FIG. 3, a peelable protective layer 6, is applied over the adhesive material of the tag attachment 1. This protective layer 6 shields the adhesive material from adhering to unintended surfaces and protects it from environmental factors such as dust, moisture, and oils to ensure the adhesive material remains effective until application. The peelable protective layer 6 is preferably composed of any substantially non-stick material such as paper, polymer, or other suitable materials and may be composed of single or multiple layers.

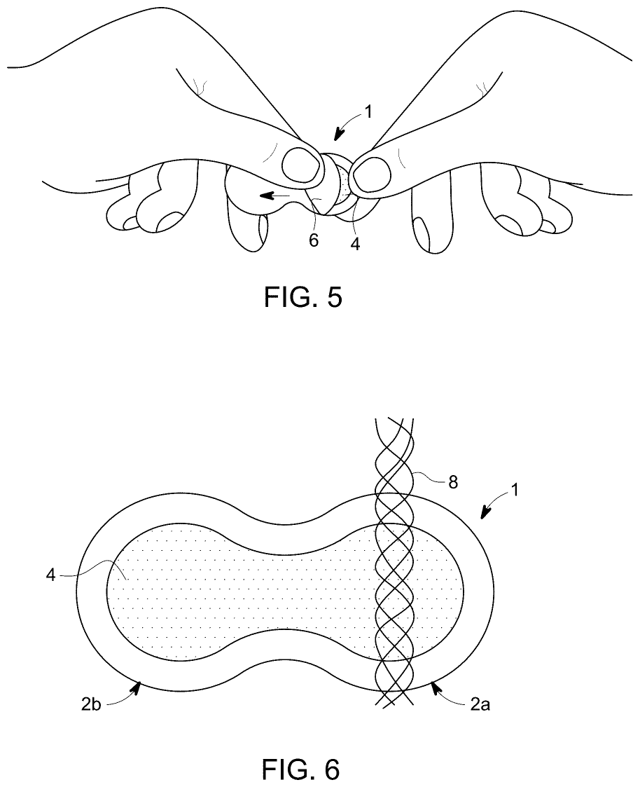

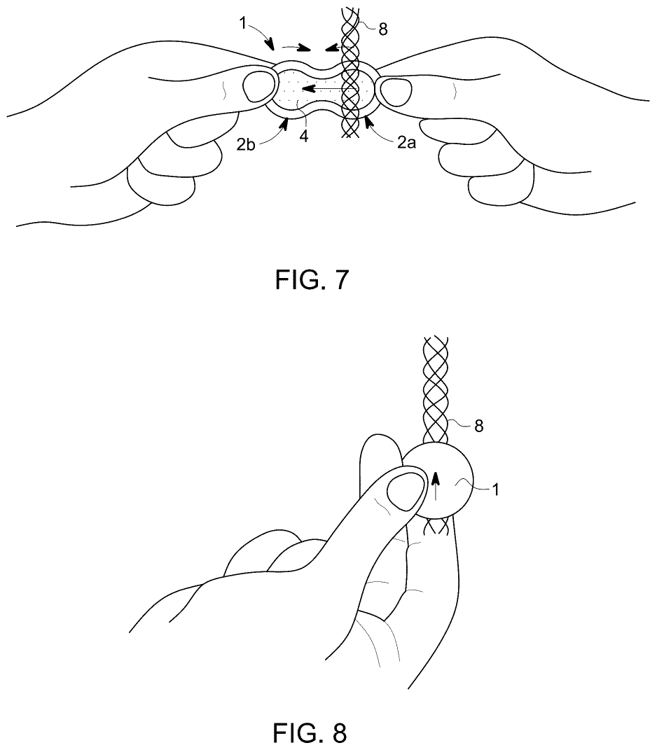

Referring now to FIG. 4, to affix the tag attachment 1 to a tampon string, the tag attachment 1 is held in one hand at the bordered edge 9, with the top side 7 facing upward. In FIG. 5, the protective layer 6 is removed to expose the adhesive material 4 on the tag attachment 1. And, as shown in FIG. 6, the user locates tampon string 8 and determines a specific point at the end of the string 8 where the tag attachment 1 will be affixed to the tampon string 8. The tampon string 8 is positioned over a first symmetrical circular part 2a, preferably centered and aligned, with the adhesive side 4 facing towards the tampon string 8. As shown in FIG. 7, using both hands as necessary, the user folds the tag attachment 1 by bringing the second symmetrical circular part 2b into contact with the first symmetrical circular part 2a, the adhesive-sided 4 of the first and second circular parts 2a and 2b brought together toward the opposite bordered edges 9 along the midsection 3, ensuring the bordered edge 9 is aligned, wherein the tampon string 8 becomes encased within the tag attachment 1. As depicted in FIG. 8, the user securely grips the tag attachment 1, holding it firmly between the thumb and the finger(s), and applies pressure to ensure a strong bond between the tampon string 8 and the adhesive material, thereby creating a secure tag attachment 1.

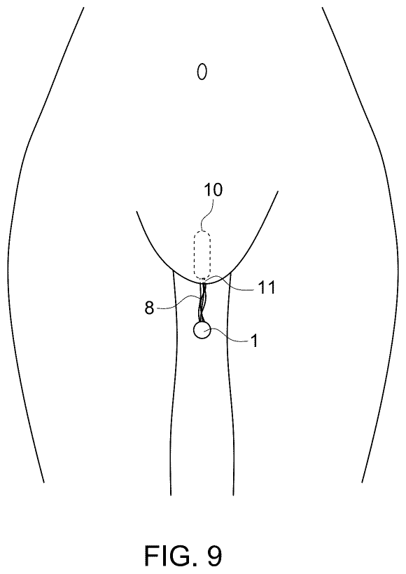

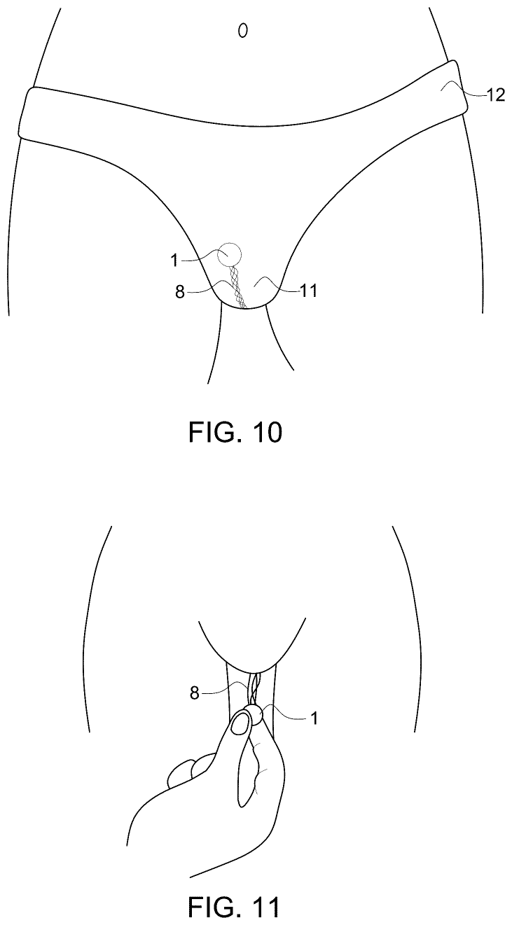

Referring now to FIG. 9, this figure illustrates a tampon 10 inserted in the vaginal canal, the tampon string 8 being secured to the tag attachment 1 as described above. Once the tampon is inserted, the user releases the grip on the tag attachment 1, allowing it to hang freely from the tampon string 8. To ensure stability and comfort, the user may position the tag attachment 1 on the outer part of the vulva 11, resting it securely on the even surface of either side of the tag attachment 1 against their pelvic area. The user then can cover the tag attachment 1 with their undergarments 12, which stabilizes the tag attachment 1 in place, preventing unintended movement or displacement as depicted in FIG. 10. This additional step helps ensure the tag attachment 1 remains securely positioned for its intended use and assists the user in remembering the tag attachment position, making it easier for them to quickly and efficiently locate it when needed for handling. FIG. 10 illustrates how the tag attachment 1 appears underneath the undergarment 12.

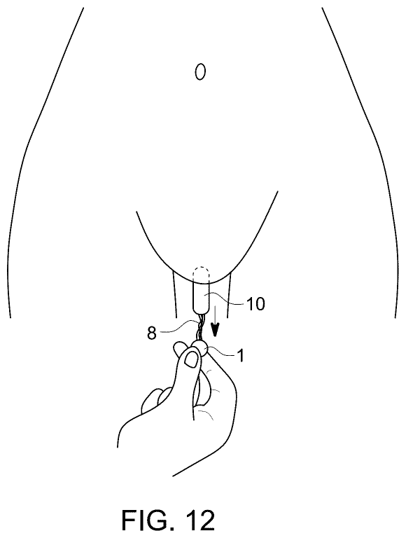

As shown in FIG. 11, to facilitate the removal of the tampon 10, the user uses their hand to locate the tag attachment 1. The user securely grips the tag attachment 1, holding it firmly between the thumb and the finger(s) on both sides and applies pressure for controlled handling. With a steady and slow motion, the user pulls the tag attachment 1 downward to guide the tampon's 10 removal from the vaginal canal as shown in FIG. 12. Once the tampon 10 is removed, the user disposes of it using their preferred disposal method.

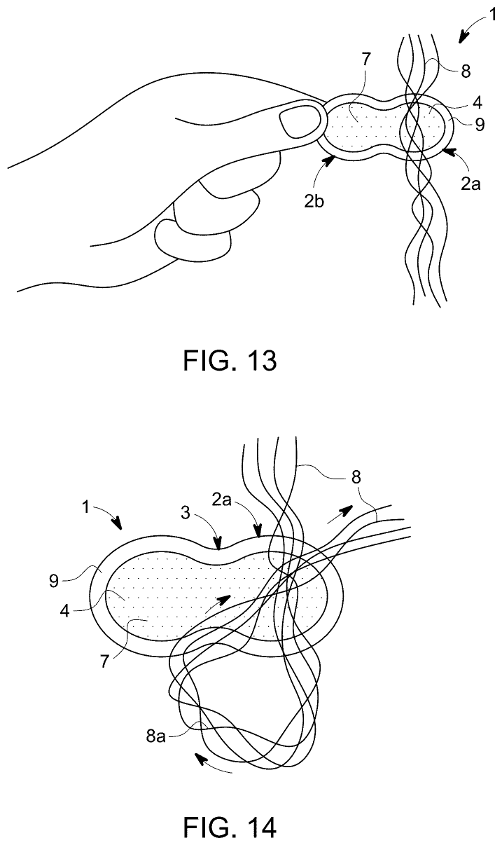

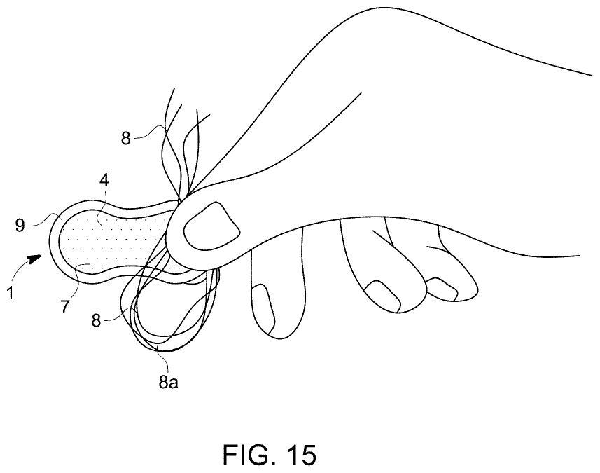

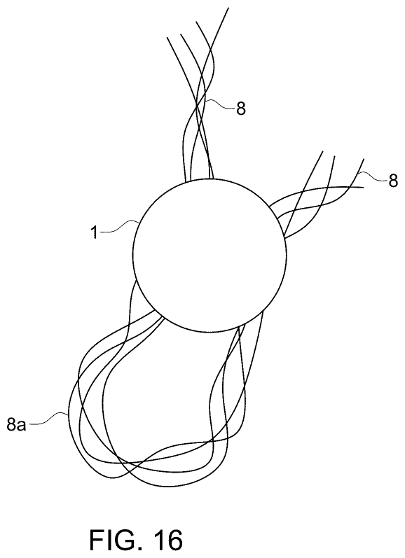

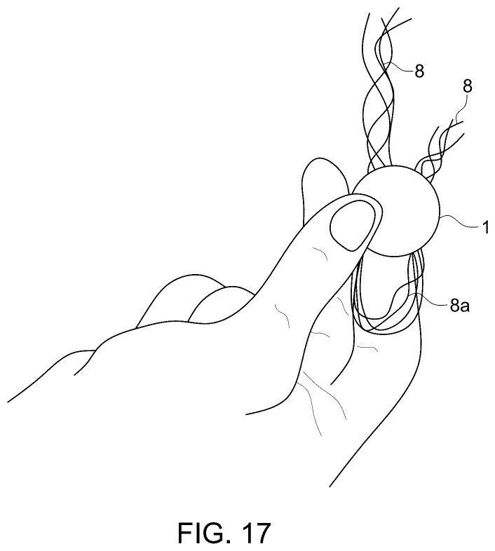

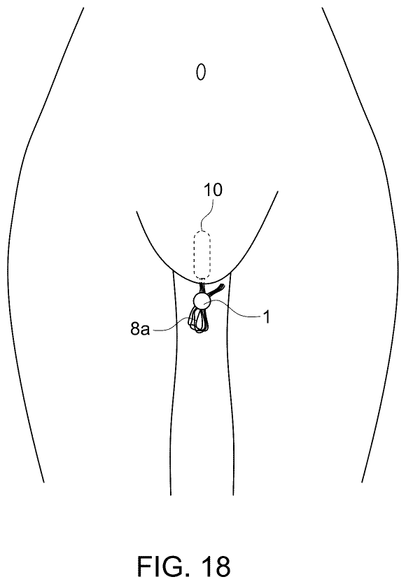

The tampon string 8 may be shortened by the user to facilitate concealment. Referring now to FIG. 13, to shorten the tampon string 8 for concealment, the tag attachment 1 is held in one hand at the bordered edge 9, with the top side 7 facing upward. The upper part of the tampon string 8 is positioned over first symmetrical circular part 2a of the tag attachment 1 with the adhesive side 4 facing toward the tampon string 8. The tampon string 8 is then adhered to the tag attachment 1. In this embodiment, the string 8 extends beyond the tag attachment 1. As illustrated in FIG. 14, using both hands as necessary, the user grips the free end of the tampon string 8 and folds it over itself to form a single loop. The user then positions the overlapping part of the tampon string 8 on the first symmetrical circular part 2a of the tag attachment 1 where the upper part of the tampon string 8 is affixed. Accordingly, FIG. 14 illustrates how the tampon string 8 appears when folded over itself to form a loop 8a. In FIG. 15, the user then secures the tampon string 8 by pressing it onto the adhesive side 4 of tag attachment 1. Then the user folds the tag attachment 1 by bringing the adhesive-side 4 of first symmetrical circular part 2a and second symmetrical circular part 2b together toward the opposite edges 9 along the midsection 3, ensuring the bordered edge 9 is aligned. FIG. 16 depicts how the user has folded the tag attachment 1, wherein the tampon string 8 becomes encased within the tag attachment 1. As shown in FIG. 17, the user securely grips the tag attachment 1, holding it firmly between the thumb(s) and the finger(s) on both sides and applies pressure to ensure a strong bond is formed between the tampon string 8 and the adhesive surface 4, creating a secure tag attachment 1. Referring now to FIG. 18, once the tag attachment 1 is affixed to the tampon string 8, the user can release the tag attachment 1 to allow it to hang freely from the vaginal canal for discreet concealment.

The tag attachment of the present subject matter solves the disadvantages typically associated with a tampon string (henceforth referred to as “string” in this section) as it extends and freely hangs outside the vaginal canal. As shown in the Figures, the tag attachment 1 is designed to be manually affixed to the end of an existing string 8 to enable the user to efficiently and effectively control and interact with the string 8. This streamlines user interaction by making it easier to locate and handle the string 8 during bathroom use to prevent it from becoming soiled with bodily fluids or to facilitate tampon 10 removal. Additionally, this design includes a feature that allows users to shorten the string 8 for discreet concealment using the tag attachment 1. The intuitive design of the tag attachment of the present subject matter focuses on usability, accessibility, functionality, and user experience by incorporating ergonomic attributes, including ample size dimensions, fibrous material composition, and the lemniscate shape. These attributes optimize ease of use, efficiency, effectiveness, inclusiveness, and comfortability, enhancing the user's ability to locate, handle, and shorten the string 8.

The tag attachment of the present subject matter is designed with ample-sized dimensions to ensure it is larger than the tampon string and differentiates the tampon string from the tag attachment so the user easily can discern the tag attachment. This provides several advantages that significantly improve usability and accessibility for the user to promptly locate and handle the tag attachment, enabling efficient grasping and maneuvering of the tampon string during bathroom use and removal of the tampon while in various positions, such as sitting or squatting over a toilet with their knees apart or standing with one leg raised.

First, the size of the tag attachment of the present subject matter makes it easily noticeable and identifiable by visual and tactile identification within the genitalia and anal areas. Its ample size is particularly useful for visually impaired users because they can easily identify it by tactile feedback. This attribute enables the user to promptly detect the location of the tag attachment to retrieve the tampon string, regardless of its position within these areas. This is advantageous as it eliminates the need for the user to employ various actions to locate the tampon string, such as re-positioning their bodies, using a mirror, or blindly searching their genitalia and anal areas by hand. Consequently, it spares the user from needing to assume awkward positions and search extensively in these areas, potentially encountering excessive bodily fluids and causing discomfort in sensitive areas.

Second, the size of the tag attachment of the present subject matter decreases the probability of it shifting or becoming obscured within the genitalia or anal areas during physical movements. Its size provides a greater surface area for friction to take effect by slowing the movement of the tag attachment between these areas. Additionally, this is particularly advantageous for the user as it prevents the tampon string from becoming displaced in the vaginal canal. It enables the user to sense the presence of the tag attachment 1 withdrawing into the canal, allowing them to promptly address the issue before it occurs. This feature alleviates concerns about the potential loss of the tampon string within the vaginal canal, reducing the need for users to seek immediate medical assistance or the help of another individual. Consequently, the size of the tag attachment stabilizes the movements of the tampon string, reducing the likelihood of it shifting or becoming nestled, withdrawn, and entangled within these areas.

Third, the size of the tag attachment of the present subject matter is designed with ample surface area for users to securely grasp it. This feature allows the user to comfortably apply pressure with their thumb and finger(s) to achieve a secure grip on the tag attachment and control the tampon string's movement for easy handling. Its ample size is exceptionally accommodating for a user with physical limitations, as it aids the user in grasping the tag attachment easily and reduces the need for precise movements. Consequently, its ample surface area eliminates the need for users to resort to various uncomfortable gripping actions to grasp the tampon string, which can cause discomfort, skin irritation on their hands, and tampon displacement. Additionally, it eliminates the need for the user to wrap or fold tissue products around the tampon string to secure a makeshift grip. This is advantageous as it allows the user to easily grasp the tag attachment, enabling reliable and effective handling to their needs and comfort level.

Moreover, the tag attachment of the present subject matter is composed of soft, lightweight, and durable fibrous material to provide an optimal balance of comfort and functionality. These attributes ensure a comfortable tactile experience and ease of handling during use and wear. The material adapts to the body's curves to provide maximum comfort, allowing the user to handle and wear the tag attachment seamlessly and comfortably. The softness of the material ensures that the tag attachment is gentle on the skin, enhancing comfort for wearability and handling. The lightweight composition enables ease of movement while ensuring it does not impede functionality. This ensures the tag attachment is suitable for extended wear and provides maximum comfort to the user.

The fibrous material is also durable enough to withstand compression under pressure from physical movements during wear or handling while ensuring the tag attachment of the present subject matter maintains its structural integrity and functionality. This feature ensures a responsive reaction to applied pressure, evenly distributing it during handling to provide a natural and comfortable grip for the user to easily manage the tag attachment in various orientations. This is advantageous as the material compresses and conforms to the thumb and finger(s) when grasped, creating a secure, non-slip grip that enables the user to grasp the tag attachment securely and effectively. Furthermore, the fibrous material may also be coated with waterproof, water-repellent, or water-resistant properties to protect against absorbing bodily fluids from the genitalia and anal areas. These properties keep the tag attachment dry for the user by repelling moisture and preventing fluid penetration, ensuring that friction between the fingers can be achieved to maintain a secure grip.

The lemniscate-shaped design of the tag attachment 1 of the present subject matter maximizes usability, accessibility, and functionality to enhance comfort and efficiency. This symmetrical lemniscate shape allows it to be evenly folded along the midsection 3 to ensure the first symmetrical circular part 2a and second symmetrical circular part 2b align. This symmetry is crucial because it ensures that the adhesive material 4 spans across the top side 7 of the circular parts 2a, 2b and encases the string 8 to create an effective, strong bond. This alignment also prevents the potential of uneven or asymmetrical bordered edge 9 that could hinder its usability and effectiveness. Moreover, the continuous curvature of the lemniscate shape enables the tag attachment 1 to rest comfortably against the body's curves, further enhancing tactile comfort during physical movement and handling. This is advantageous as it ensures the tag attachment 1 can be worn comfortably and discreetly for extended periods. Its sleek, uniform, and compact form remains unnoticeable, minimizing the likelihood of it being felt or creating visible bulges under clothing when worn. Additionally, the lemniscate shape complements the natural curves of the hands to provide a comfortable and secure grip for the user to effortlessly and effectively handle the tag attachment 1 in various orientations.

The tag attachment 1 is designed to be affixed at the end of the tampon string 8 to maximize the ergonomic attributes and enable the user to handle and control the string's 8 movements efficiently and effectively. This placement is key to allowing the user to precisely control the tampon's string's 8 movements and efficiently maneuver it, such as positioning and adjusting it, in various directions without directly touching it. This is advantageous for the user to effectively position the string 8 away from bodily fluids during bathroom use to prevent it from becoming soiled and to remove the tampon 10 from the vaginal canal. Additionally, it ensures that the ergonomic attributes significantly enhance the user's interaction with the string 8, facilitating an intuitive and comfortable experience.

Using the tag attachment 1 to handle and control the string 8 during bathroom use provides significant advantages for the user, particularly for the user who prefers to keep the tampon 10 inserted to avoid needing to change it. The tag attachment 1 enables the user to easily position and maneuver the string 8 away from the urethra and anus openings to prevent direct contact with streaming fluids such as urination and defecation. This ensures optimal hygiene by ensuring the string 8 remains clean and dry for continued wear. Additionally, the tag attachment 1 enables the user to maneuver the string 8 to a position that prevents direct contact with the genitalia and anal areas. This is particularly advantageous for the user who prefers minimal contact with touching these areas when handling the string 8. It also enables the user to maneuver the tampon string 8 effortlessly to avoid contact with sensitive areas and skin conditions to prevent irritation, aggravation, and discomfort while ensuring user comfort.

Moreover, handling and controlling the movements of the tampon string 8 allows the user to manage the amount of force applied effectively. This reduces the likelihood of unintended pulling being applied to the tampon string 8 during handling, which prevents the tampon 10 from shifting downward and becoming displaced in the vaginal canal or leading to premature removal. This is advantageous for the user who needs to remove their tampons 10. The tag attachment 1 allows the user to adjust the position of the tampon string and manage it effectively to ensure the tampon 10 remains in place until the user is ready to remove it from the vaginal canal. This allows the user to comfortably position the string 8 and then apply the necessary force to pull on it to remove the tampon 10.

Consequently, effectively handling and controlling the movements of the tampon string eliminates the need for the user to resort to various actions to handle it. It empowers the user to grasp and control the string 8 effectively, minimizing the need to change their body positions or adopt uncomfortable postures for string 8 manipulation. With the tag attachment 1, the user can manage the string 8 comfortably while maintaining balance. It also prevents the user from needing to resort to placing the string 8 in the vaginal canal or tucking it between the skin folds of the labia to keep it dry during bathroom use. This eliminates the need to tuck the tampon string 8 inside the vaginal canal, alleviating any concerns about forgetting it and avoiding the risk of toxic shock syndrome.

Moreover, the tag attachment 1 enables the user to shorten the length of the tampon string 8 for discreet concealment during wear. The user can adjust the length of the tampon string 8 to the user's preference by simply looping it and affixing it to the tag attachment 1. This is advantageous as it provides a simple and efficient process to shorten the string's 8 length, ensuring it remains concealed beneath undergarments to prevent unintended exposure. Consequently, it eliminates the need for the user to employ complex techniques such as cutting, knotting, or tucking the string 8 to shorten it.

Further, the tag attachment of the present subject matter is thoughtfully designed with ergonomic considerations, take into account its size, material, and shape to ensure accessibility for users with physical limitations. These attributes enable the user to locate, grasp, and maneuver the tag attachment 1 effectively, facilitating efficient handling and shortening of the string 8 with minimal effort. This enhances overall usability and functionality for all users.

In conclusion, the tag attachment of the present subject is a significant advancement in addressing the disadvantages of a tampon string. It greatly enhances user interaction by simplifying the process of locating, handling, and shortening a tampon string efficiently and effectively. The incorporation of ergonomic attributes emphasizes usability, accessibility, functionality, and user experience to ensure an effortless experience for users. The tag attachment is pivotal in improving feminine hygiene products.

While several illustrative embodiments of the invention have been shown and described, numerous variations and alternative embodiments will occur to those skilled in the art. Such variations and alternative embodiments are contemplated, and can be made without departing from the scope of the invention as defined in the appended claims.

As used in this specification and the appended claims, the singular forms “a,” “an,” and “the” include plural referents unless the content clearly dictates otherwise. The term “plurality” includes two or more referents unless the content clearly dictates otherwise. Unless defined otherwise, all technical and scientific terms used herein have the same meaning as commonly understood by one of ordinary skill in the art to which the disclosure pertains.

The foregoing detailed description of exemplary and preferred embodiments is presented for purposes of illustration and disclosure in accordance with the requirements of the law. It is not intended to be exhaustive nor to limit the invention to the precise form(s) described, but only to enable others skilled in the art to understand how the invention may be suited for a particular use or implementation. The possibility of modifications and variations will be apparent to practitioners skilled in the art. No limitation is intended by the description of exemplary embodiments which may have included tolerances, feature dimensions, specific operating conditions, engineering specifications, or the like, and which may vary between implementations or with changes to the state of the art, and no limitation should be implied therefrom. Applicant has made this disclosure with respect to the current state of the art, but also contemplates advancements and that adaptations in the future may take into consideration of those advancements, namely in accordance with the then current state of the art. It is intended that the scope of the invention be defined by the Claims as written and equivalents as applicable. Reference to a claim element in the singular is not intended to mean “one and only one” unless explicitly so stated. Moreover, no element, component, nor method or process step in this disclosure is intended to be dedicated to the public regardless of whether the element, component, or step is explicitly recited in the claims. No claim element herein is to be construed under the provisions of 35 U.S.C. Sec. 112, sixth paragraph, unless the element is expressly recited using the phrase “means for . . . ” and no method or process step herein is to be construed under those provisions unless the step, or steps, are expressly recited using the phrase “step(s) for. . . . ”

What is claim is:

1. A tag attachment having a lemniscate shape which can be securely attached to the tampon string of a tampon by encasing said tampon string within said tag attachment.

2. The tag attachment in accordance with claim 1, wherein said tag attachment consists of a top surface, a bordered edge, a first symmetrical circular part and a second symmetrical circular part, said first symmetrical circular part being connected to said second symmetrical circular part by a flexible midsection.

3. The tag attachment in accordance with claim 2, wherein said top surface is provided with a layer of adhesive material which does not extend into the bordered edge, and a peelable and removable protective layer which is applied over said layer of adhesive material.

4. The tag attachment in accordance with claim 3, wherein said adhesive material is selected from the group consisting of pressure-sensitive adhesives and polyolefin-based hot melt adhesives.

5. The tag attachment in accordance with claim 3, wherein when said peelable and removable protective layer is removed, said layer of adhesive material is exposed and said tampon string is positioned over said first symmetrical circular part and onto said layer of adhesive and said second symmetrical circular part is folded at said flexible midsection in such a manner that said second symmetrical circular part is folded onto said first symmetrical circular part and adhesively secured thereto, thereby encasing said tampon string within said tag attachment.

6. The tag attachment in accordance with claim 5, wherein the positioning of said tampon string is centered and aligned over said first symmetrical circular part.

7. The tag attachment in accordance with claim 5, wherein said second symmetrical circular part is folded onto said first symmetrical circular part in such a manner that the bordered edge of said lemniscate-shaped tag attachment is in alignment.

8. The tag attachment in accordance with claim 5, wherein pressing the folded said first symmetrical circular part and said second symmetrical circular part together adhesively secures said first symmetrical circular part to said second symmetrical circular part, said tampon string being firmly encased within said tag attachment.

9. The tag attachment in accordance with claim 6, wherein once said tampon string is firmly encased within said tag attachment, the tampon can be introduced into a user's vagina and said tag attachment can be positioned in a user's underwear, away from the user's vulva area.

10. The tag attachment in accordance with claim 9, wherein when a user pulls the tag attachment downwardly, the tampon is dislodged from the vagina.

11. The tag attachment in accordance with claim 5, wherein the length of said tampon string can be shortened by folding said tampon string over and onto itself to create a single tampon string loop extending outside the tag attachment, the length of the tampon string not forming said tampon string loop being encased within said tag attachment.

12. The tag attachment in accordance with claim 1, wherein said tag attachment is composed of a fibrous material that is characterized as being soft, lightweight, and durable.

13. The tag attachment in accordance with claim 12, wherein said fibrous material is selected from the group consisting of cotton, bamboo, cellulose made from wood pulp and lyocell.

14. The tag attachment in accordance with claim 12, wherein said fibrous material is waterproof, water-repellant, or water-resistant.

15. The tag attachment in accordance with claim 12, wherein said fibrous material is coated with a substance that is waterproof, water-repellant, or water-resistant.

16. The tag attachment in accordance with claim 5, wherein said tag attachment is composed of a fibrous material that is characterized as being soft, lightweight, and durable.

17. The tag attachment in accordance with claim 16, wherein said fibrous material is selected from the group consisting of cotton, bamboo, cellulose made from wood pulp and lyocell.

18. The tag attachment in accordance with claim 16, wherein said fibrous material is waterproof, water-repellant, or water-resistant.

19. The tag attachment in accordance with claim 16, wherein said fibrous material is coated with a substance that is waterproof, water-repellant, or water-resistant.

20. The tag attachment in accordance with claim 1, wherein said tag attachment is about one-and-one-half inches (1½″) in length and about one half inch (½″) in height.
